(19) United States
(12) Patent Application Publication
(10) Pub. No.: US 2015/0203916 A1
(43) Pub. Date: Jul. 23, 2015

(54) PLASMA ANALYTES PREDICT DIAGNOSIS AND PROGNOSIS OF THORACIC AORTIC ANEURYSM

(71) Applicant: MUSIC FOUNDATION FOR RESEARCH DEVELOPMENT, Charleston, SC (US)

(72) Inventors: John S. Ikonomidis, Mount Pleasant, SC (US); Jeffrey A. Jones, Charleston, SC (US); Mukherjee Rupak, Charleston, SC (US); Robert E. Stroud, Mount Pleasant, SC (US); Michael R. Zile, Charleston, SC (US)

(21) Appl. No.: 14/411,126

(22) PCT Filed: Jun. 27, 2013
(86) PCT No.: PCT/US2013/048280

§ 371 Date: Dec. 24, 2014

Related U.S. Application Data
(60) Provisional application No. 61/664,863, filed on Jun. 27, 2012.

(51) Int. Cl. C12Q 1/68; A61B 6/03; A61B 5/055; G01N 33/573; G01N 33/68

(52) U.S. Cl. CPC C12Q 1/6883; G01N 33/573; G01N 33/6893; A61B 5/055; A61B 6/03; C12Q 2600/178; C12Q 2600/16; C12Q 2600/118; G01N 2333/96486; G01N 2333/8146; G01N 2800/329

(57) ABSTRACT

Disclosed are methods and materials for assessing thoracic aortic aneurysm using a combination of protein and microRNA biomarkers. The presence or levels of the biomarkers can be measured in a body fluid, such as plasma and serum, or in cardiac tissue, to predict the presence and severity of TAA in a subject. This can be used to diagnose and monitor TAA, providing early detection of a lethal and silent disease, as well as reduce the frequency of radiological procedures, which are costly and potentially dangerous.

### CROSS-REFERENCE TO RELATED APPLICATIONS

This application claims benefit of U.S. Provisional Application No. 61/664,863, filed Jun. 27, 2012, which is hereby incorporated herein by reference in its entirety.

### STATEMENT REGARDING FEDERALLY SPONSORED RESEARCH OR DEVELOPMENT

This invention was made with Government Support under Agreements R21 HL089170-01A1, R01 HL102121-01A1, R01 AG036954-01A1 awarded by the National Institutes of Health; and Agreements I01 BX000904-01 and I01 BX000904-01awarded by the Veterans Administration. The Government has certain rights in the invention.

### TECHNICAL FIELD

The disclosed technology is generally in the field of cardiac disease and cardiac failure and specifically in the area of diagnosis, prognosis, and monitoring of thoracic aortic aneurysm (TAA).

### BACKGROUND

The incidence of thoracic aortic aneurysm (TAA) disease doubled between 1982 and 2002. Current projections suggest, with the aging of the “Baby Boomer” generation, that the number of patients diagnosed and living with aneurysms is likely to rise significantly in the coming years. Patients often remain asymptomatic, resulting in dilation and possible rupture, and are usually diagnosed serendipitously during a routine physical examination or work-up for another medical issue. At present, the diagnosis of aneurysm disease is highly dependent on costly advanced imaging techniques using primarily computed tomography (CT) and magnetic resonance (MRI). At present there are no point-of-care plasma biomarker assays available that can be used to diagnose TAA or follow disease progression to inform optimal timing for surgical intervention. Once diagnosed, a “watch and wait” surveillance program is initiated until the risk of aortic rupture outweighs the risk of the surgical repair. While recent advancements such as endovascular stent-grafting have significantly decreased the early mortality and postoperative complications associated with open surgical procedures, the complication rates remain high, and similar to open procedures, neither approach is aimed directly at the underlying cellular and molecular mechanisms responsible for this devastating disease.

### SUMMARY

Disclosed are methods and materials for assessing thoracic aortic aneurysm (TAA) using biomarkers that include microRNAs (miRs), matrix metalloproteinases (MMPs), and tissue inhibitors of MMPs (TIMPs). In particular, a combination of specific MMPs, TIMPs, and miRs can be used for diagnosing and predicting the severity of TAA in a subject. The levels of the biomarkers can be measured in a body fluid, such as plasma and serum, or in tissue, such as cardiac and aortic tissue. The levels of the biomarkers can provide a biomarker profile that is used to compare to the same biomarker profile in control samples. As disclosed herein, level of the biomarker combinations indicates, for example, the risk, development, presence, severity, or a combination, of TAA in the subject.

Methods of treating TAA, such as ascending TAA, in a patient are disclosed. These method can involve assaying a blood or plasma sample from a subject diagnosed with TAA for levels of microRNAs, MMPs, TIMPs, or a combination thereof, comparing the levels to control values to predict the aneurysm size or by multivariate logistic regression analysis, to determine the probability of having a TAA, and selecting a course of treatment for the patient based on the presence and predicted aneurysm size. Based on analyte levels, aneurysm size can be estimated. The relationship between aneurysm size and levels of microRNAs, MMPs, TIMPs, or a combination thereof, from TAA as compared to control values, can be determined using regression analysis. For example, in BAV patients with aneurysms the levels of MMP-2 are in direct relationship to aneurysm size, while MMP-3, MMP-14, and TIMP-1 are in an inverse relationship to aneurysm size. In TAV patients with aneurysms, the levels of MMP-7 and MMP-13 are in direct relationship to aneurysm size, while the levels of MMP-13, TIMP-2, miR-1, miR-21, miR-29a, and miR-133a are in an inverse relationship to aneurysm size.

In some embodiments of these methods, the course of treatment involves imaging the patient to measure the size of the aneurysm if the levels predict that the aorta is at least about 4 to 6 centimeters (cm) in size. Patients may be imaged by any method suitable to detect and measure TAAs in vivo. For example, the patient may be imaged by computed tomography (CT), magnetic resonance imaging (MRI), or a combination thereof to measure the size of the aneurysm.

The size of the aneurysm in addition to a patient's body surface area (BSA; calculated as BSA (m²)=0.20247*((weight(kg)^{0.425})*(height(m))^{0.725}) plays an important role in the decision for surgery. While 5.0 cm is the size most aneurysms are considered for surgery, a patient's BSA is considered and their aneurysm size is adjusted to generate an Aortic Size Index (ASI; cm/m²) which strongly correlates with the need for surgery due to rupture risk (less than 2.75 cm/m² are at low risk, 2.75 to 4.24 cm/m² are at moderate risk, and greater than 4.25 cm/m² are at high risk). For instance, a patient with an BSA of (2.50) with a 7.5 cm aneurysm would have an ASI of 3.00 and would be recommended for surgery due to a moderately high risk for aortic rupture. Yet, a patient who has a BSA of 1.30 and a thoracic aneurysm of 4.0 cm (ASI=3.08) would also be a candidate for surgery due to a similar individual risk of rupture. Therefore, once the patient has been imaged and the TAA has been confirmed to be at least about 4 to 6 cm in size, depending on other clinical considerations, the course of treatment may be surgical treatment of the TAA. This generally involves the replacement of the diseased portion of the aorta with a fabric tube or graft (e.g., Dacron® graft) or placement of a stent graft. However, if the TAA is present and the ASI is less than 2.75, then the course of treatment may be to continue monitoring levels on a weekly, biweekly, monthly, bimonthly, quarterly, semi-annual, or annual basis.

Control values can be obtained from different sources depending on the method being used. In some embodiments, the control values are based on one or more of a) levels obtained from a bodily fluid sample from a healthy subject or b) levels obtained from a bodily fluid sample from a diseased subject, e.g., having a TAA at least about 5 cm in size. These values can be obtained in advance and provided as reference values or obtained in parallel using control samples. In other embodiments, the control values are levels obtained from a bodily fluid sample from the patient at an earlier time point. In these embodiments, the method involves comparing changes in levels over time.

Specific MMPs, TIMPs, and miRs are shown herein to be differentially expressed in subjects with TAA. In some embodiments, the microRNA assayed in the disclosed methods may be selected from the group consisting of miR-455-3p, miR-1268, miR-338-5p, miR-940, miR-1323, miR-768-3p, miR-574-3p, miR-106b, miR-451, miR-100, miR-125b, miR-195, miR-19b, miR-30d, miR-15b, miR-125a-5p, miR-143, miR-193b, miR-16, miR-27a, miR-29a, miR-30a, miR-27b, miR-92a, miR-140-5p, let-7i, miR-151-5p, miR-140-3p, miR-24, miR-23a, miR-145, miR-199b-3p, miR-199a-3p, miR-361-5p, miR-130a, miR-22, and miR-497. In some embodiments, the microRNA assayed in the disclosed methods may be selected from the group consisting of miR-1, miR-21, miR-29a, miR-133a, miR-143, and miR-145. In some embodiments, the MMP assayed in the disclosed methods may be selected from the group consisting of MMP-1, MMP-2, MMP-3, MMP-7, MMP-8, MMP-9, MMP-12, MMP-13, MMP-14, MMP-15, MMP-16, MMP-17, MMP-19, MMP-20, MMP-21, MMP-23a, MMP-23b, MMP-24, MMP-25, MMP-26, MMP-27, and MMP-28.

In some embodiments, the MMP assayed in the disclosed methods may be selected from the group consisting of MMP-1, MMP-2, MMP-3, MMP-7, MMP-8, MMP-9, MMP-12 and MMP-13. In some embodiments, the TIMP assayed in the disclosed methods may be selected from the group consisting of TIMP-1, TIMP-2, TIMP-3, and TIMP-4.

Moreover, disclosed is a method of diagnosing TAA, such as ascending TAA, in a patient that involves assaying a blood or plasma sample from the patient for the levels of microRNAs, MMPs, TIMPs, or a combination thereof, and determining the probability that the patient has a TAA using multivariate logistic regression analysis. The method can further involve imaging the patient to verify the presence and severity of the aneurysm if the levels indicate that the patient has a TAA. For example, in some embodiments, the combination of miR-143, MMP-8, and miR-133a levels are analyzed together to determine the presence of aneurysm. In some embodiments, the combination of MMP-2, miR-143, and MMP-8 levels are analyzed together to determine the presence of aneurysm in a subject that has a tricuspid aortic valve. In some embodiments, the combination of MMP-2, TIMP-2, miR-143, miR-133a, and miR-145 levels are analyzed together to determine the presence of aneurysm in a subject that has a bicuspid aortic valve.

Using a multivariate logistic regression equation, such as the equation disclosed in the examples, a predictability value can be calculated. In some embodiments, any value greater than 0 could suggest the presence of an aneurysm. Any value less than or equal to 0 could suggest the absence of an aneurysm. For example, the cutoff can be chosen to reduce false negatives so that all aneurysm patients would be identified (in theory) and would indicate the need for an advanced imaging series to precisely determine aneurysm presence and size.

Also disclosed is a method for monitoring the efficacy of a therapeutic agent in the treatment of TAA, such as ascending TAA, in a subject. This method can involve treating the subject with the therapeutic agent during a treatment period, assaying blood or plasma samples from the subject at two or more intervals during the treatment period for the levels of microRNAs, MMPs, TIMPs, or a combination thereof, and comparing changes to the levels over the course of treatment by multivariate analysis to determine whether the therapeutic agent is effectively treating the TAA. For example, in some embodiments, the combination of miR-143, MMP-8, and miR-133a levels are analyzed together by multivariate analysis. In some embodiments, the combination of MMP-2, miR-143, and MMP-8 levels are analyzed together by multivariate analysis if the subject has a tricuspid aortic valve. In some embodiments, the combination of MMP-2, TIMP-2, miR-143, miR-133a, and miR-145 levels are analyzed together by multivariate analysis if the subject has a bicuspid aortic valve.

Also disclosed is a system for diagnosing or predicting thoracic aortic aneurysm (TAA), such as ascending TAA. This system can include an immunoassay for detecting levels of one or more MMPs and/or TIMPs. For example, the immunoassay can be a lateral flow immunoassay comprising one or more antibodies that selectively bind two or more MMPs and/or TIMPs, such as those disclosed herein. The system can also include nucleic acid primers or probes for detecting levels of one or more microRNAs, such as those disclosed herein. For example, the system can contain quantitative RT-PCT (qRT-PCR) primer sets and reagents for detecting one or more microRNAs.

Specific plasma signatures have also been identified which are predictive of the presence and/or size of TAA. For example, in some embodiments, an at least two-fold decrease in MMP-3 and microRNA-29a levels compared to the control values indicates an increase in aneurysm size if the subject has a tricuspid aortic valve. In some embodiments, an at least two-fold decrease in MMP-1, MMP-2, MMP-3, MMP-8, MMP-9, TIMP-1, TIMP-2, TIMP-4, and microRNA-29a levels compared to the control values indicates an increase in aneurysm size if the subject has a tricuspid aortic valve. In some embodiments, an at least two-fold increase in MMP-1 levels and an at least two-fold decrease in TIMP-3 and microRNA-133a levels compared to the control values indicates an increase in aneurysm size if the subject has a bicuspid aortic valve. In some embodiments, an at least two-fold increase in MMP-1 levels and an at least two-fold decrease in MMP-2, MMP-8, MMP-9, TIMP-1, TIMP-2, TIMP-3, TIMP-4, and microRNA-133a levels compared to the control values indicates an increase in aneurysm size if the subject has a bicuspid aortic valve.

Also disclosed is a method of diagnosing TAA, such as ascending TAA, in a patient using the disclosed plasma signatures. This can involve assaying a blood or plasma sample from a patient for the levels of microRNAs, MMPs, and TIMPs, wherein an at least two-fold decrease in MMP-3 and microRNA-29a levels compared to the control values indicates the presence of a TAA if the patient has a tricuspid aortic valve, and wherein an at least two-fold increase in MMP-1 levels and an at least two-fold decrease in TIMP-3 and microRNA-133a levels compared to the control values indicates the presence of a TAA if the patient has a bicuspid aortic valve. In some embodiments of this method, an at least two-fold decrease in MMP-1, MMP-2, MMP-3, MMP-8, MMP-9, TIMP-1, TIMP-2, TIMP-4, and microRNA-29a levels compared to the control values indicates the presence of a TAA if the subject has a tricuspid aortic valve. In some embodiments of this method, an at least two-fold increase in MMP-1 levels and an at least two-fold decrease in MMP-2, MMP-8, MMP-9, TIMP-1, TIMP-2, TIMP-3, TIMP-4, and microRNA-133a levels compared to the control values indicates the presence of a TAA if the subject has a bicuspid aortic valve. In some embodiments of this method, an at least two-fold increase in microRNA-142, microRNA-140, and microRNA-128-1 levels and an at least two-fold decrease in microRNA-345 levels compared to the control values indicates the presence of a TAA if the subject has a bicuspid aortic valve.

Also disclosed is a method for monitoring the efficacy of a therapeutic agent in the treatment of TAA, such as ascending TAA, in a subject using the disclosed plasma signatures. This can involve treating the subject with the therapeutic agent during a treatment period, and assaying blood or plasma samples from the subject at two or more intervals over the treatment period for the levels of microRNAs, MMPs, TIMPs, or a combination thereof. In some embodiments, an increase in MMP-3 and microRNA-29a levels over the course of treatment indicates that the therapeutic agent is effectively treating the TAA if the subject has a tricuspid aortic valve, and wherein an decrease in MMP-1 levels and an increase in TIMP-3 and microRNA-133a levels compared to the control values indicates that the therapeutic agent is effectively treating the TAA if the subject has a bicuspid aortic valve. In some embodiments, an at least two-fold increase in MMP-1, MMP-2, MMP-3, MMP-8, MMP-9, TIMP-1, TIMP-2, TIMP-4, and microRNA-29a levels compared to the control values indicates that the therapeutic agent is effectively treating the TAA if the subject has a tricuspid aortic valve. In some embodiments, an at least two-fold decrease in MMP-1 levels and an at least two-fold increase in MMP-2, MMP-8, MMP-9, TIMP-1, TIMP-2, TIMP-3, TIMP-4, and microRNA-133a levels compared to the control values indicates that the therapeutic agent is effectively treating the TAA if the subject has a bicuspid aortic valve.

In some embodiments, an at least two-fold decrease in microRNA-142, microRNA-140, and microRNA-128-1 levels and an at least two-fold increase in microRNA-345 levels compared to the control values indicates that the therapeutic agent is effectively treating the TAA if the subject has a bicuspid aortic valve. In some embodiments, an at least two-fold increase in microRNA-142, microRNA-140, and microRNA-128-1 levels and an at least two-fold decrease in microRNA-345 levels compared to the control values indicates an increase in aneurysm size if the subject has a bicuspid aortic valve.

Additional advantages of the disclosed methods and compositions will be set forth in part in the description which follows, and in part will be understood from the description, or may be learned by practice of the disclosed method and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### DESCRIPTION OF DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B and 1C show bar graphs of the percent of referent normals versus different biomarkers in either tissue or plasma. The aortic tissue and plasma analysis for miRs, MMPs and TIMPs, comparing ascending TAAs associated with BAV or TAV from normal aortic samples (dashed line) is shown. Significant differences were observed between the BAV and TAV groups, and between the aneurysm groups and normal aorta. *p<0.05 from normal aorta, #p<0.05 from BAV.

FIG. 2 shows the linear regression analysis of plasma levels (pg/mL) versus tissue levels (pg/mL). The analysis identified significant relationships between analyte tissue and plasma levels. Significant relationships were found for MMP-8, TIMP-1, TIMP-3 and TIMP-4.

FIG. 3 shows receiver operating characteristic curves of sensitivity versus specificity. The receiver operating characteristic curves assess plasma aneurysm predictability. Inclusion of plasma analytes using forward stepwise variable selection resulted in different combinations for TAA in general (top), BAV-associated TAAs (middle) and TAV-associated TAAs (bottom) providing high area-under-the curve (AUC) values, indicating high sensitivity and specificity.

FIG. 4 is a bar graph of percent change of referent controls versus different MMP biomarkers. The relative proteolytic balance expressed as the ratio of MMP abundance to a composite TIMP score composed of the sum of TIMP-1, TIMP-2, TIMP-3, and TIMP-4. Different profiles of proteolytic balance were observed for the BAV and TAV groups. *p<0.05 from normal aorta.

FIG. 5 is diagram depicting number of miRs differentially expressed at least 2-fold in BAV vs. Control, BAV-TAA vs. BAV, and BAV-TAA vs. Control.

### DETAILED DESCRIPTION

Within the spectrum of cardiovascular diseases, TAAs continue to be one of the most dangerous and difficult to treat problems in cardiothoracic surgery. TAA development is influenced by a series of interrelated mechanisms that result in a weakened aortic wall and gross dilatation progressing to rupture if left untreated. There are numerous etiologies of TAA, with the most common type being related to idiopathic medial degeneration in patients with tricuspid aortic valves (TAV). Other etiologies include TAAs that form secondary to connective tissue disorders, such as Marfan syndrome (MFS), or congenital cardiovascular malformations such patients that possess a bicuspid aortic valve (BAV). Identification of the etiological sub-type of aneurysm disease is essential as it factors into surgical decision making tree. As disclosed herein, one or more combinations of microRNAs (miRs), matrix metalloproteinases (MMPs), and tissue inhibitors of matrix metalloproteinases (TIMPs) can be used as biomarkers for TAA diagnosis, prognosis, etiology, or monitoring.

The singular forms “a”, “an”, and “the” include plural reference unless the context clearly dictates otherwise. Thus, for example, reference to “a microRNA” includes a plurality of such microRNAs, reference to “the microRNA” is a reference to one or more microRNAs and equivalents thereof known to those skilled in the art, and so forth.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

“Treatment” or “treating” or like terms refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Independent of etiology, it has become clear that aortic dysfunction and dilatation are a direct result of pathological remodeling of the aortic extracellular matrix (ECM), and that this process is a result of an imbalance between matrix deposition and matrix degradation characterized by a significant spatiotemporal change in the expression/abundance of the matrix metalloproteinases (MMPs) and their endogenous tissue inhibitors (TIMPs). Aortic tissue specimens from patients with ascending TAAs and BAVs, TAVs, and MFS have unique profiles of MMP/TIMP protein abundance.

MMPs are zinc-dependent endopeptidases; other family members are adamalysins, serralysins, and astacins. The MMPs belong to a larger family of proteases known as the metzincin superfamily. The MMPs share a common domain structure. The three common domains are the pro-peptide, the catalytic domain and the haemopexin-like C-terminal domain which is linked to the catalytic domain by a flexible hinge region. The MMPs can be subdivided in different ways. Use of bioinformatic methods to compare the primary sequences of the MMPs indicates the following evolutionary groupings of the MMPs: MMP-19; MMPs 11, 14, 15, 16 and 17; MMP-2 and MMP-9; all the other MMPs. As disclosed herein, MMPs can be used in combination with each other and with other biomarkers in the disclosed methods and as indicators of TAA. MMPs can be combined with each other and with any other biomarker or combination of biomarkers.

MMPs are inhibited by specific endogenous tissue inhibitor of metalloproteinases (TIMPs), which comprise a family of four protease inhibitors: TIMP-1, TIMP-2, TIMP-3 and TIMP-4. Overall, all MMPs are inhibited by TIMPs once they are activated but the gelatinases (MMP-2 and MMP-9) can form complexes with TIMPs when the enzymes are in the latent form. The complex of latent MMP-2 (pro-MMP-2) with TIMP-2 serves to facilitate the activation of pro-MMP-2 at the cell surface by MT1-MMP (MMP-14), a membrane-anchored MMP. As disclosed herein, TIMPs can be used in combination with each other and with other biomarkers in the disclosed methods and as indicators of TAA. TIMPs can be combined with each other and with any other biomarker or combination of biomarkers.

In addition, microRNAs (miRs), 20-25 nucleotides in length, have important post-transcriptional gene regulatory functions. miRs are noncoding RNAs that bind to target mRNAs and reduce their expression through translational repression or mRNA degradation. Measurements made in myocardial tissue have indicated that miRs can have a predictive value in cardiovascular diseases, such as left ventricular hypertrophy and myocardial infarction. However, miRs in the plasma have not previously been linked to TAA and neither has the combination of miRs with other biomarkers such as MMPs and TIMPs. As disclosed herein, miRs can be used in combination with each other and with other biomarkers in the disclosed methods and as indicators of TAA. miRs can be combined with each other and with any other biomarker or combination of biomarkers.

MicroRNAs target short nucleotide sequences within the 3′ untranslated region (UTR) of specific messenger RNAs (mRNAs), and function to induce message degradation, or more typically, translational repression. To date, more than 1,000 unique miRs have been identified within the human genome (miRBase statistics), and based on computational methodology current predictions suggest that approximately one third of expressed human genes contain miR regulatory target sites. The examination of miR expression in tissue specimens from patients with ascending TAAs and TAVs have identifed multiple miRs that change expression level in an inverse relationship to the change in aortic diameter. Moreover, specific miR expression is also shown to be inversely proportional to specific MMP abundance.

As disclosed herein, a combination of MMPs, TIMPs, and miRs can be used to diagnosis or predict TAA or a TAA subtype. The combination can include one or more biomarkers from two or three of the groups or can include multiple biomarkers from one group. The analysis of the different combinations of biomarkers results in specific profiles of plasma analytes that can be predictive of the presence of TAA disease, and can allow for the differentiation between etiological TAA subtypes (TAAs derived from idiopathic medial degeneration in patients with TAV versus TAAs that arise in patients that possess a BAV).

Furthermore, these analytes can be measured in combination with other key circulating proteins and peptides (e.g. transforming growth factor-beta, SPARC, and collagen pro- and telo-peptides) to further refine a predictive plasma profile panel. Plasma levels of MMPs, TIMPs, and miRs can be reliably measured thereby providing a pathway for diagnostic and prognostic use. In addition, the identification of specific MMPs, TIMPs, and miRs in relevant TAA disease states, can further identify unique pharmacological targets for specific intervention, holding significant relevance for drug discovery in pharmaceutical industry and personalized medicine.

The combination of biomarkers can include two or more biomarkers from the group of miRs, MMPs and TIMPs. In some embodiments, the two or more markers are not all from the same group. In some forms, at least one of the two or more biomarkers can be miR-133a, miR-143, miR-145, MMP-2, MMP-8, or TIMP-2. In some forms, at least one of the two or more biomarkers can be miR-142, miR-345, miR-140, miR-128-1.

The combination of biomarkers can be measured together or separately. Regardless of whether they are measured together or not, it is the combination of the increase or decrease of the different biomarkers that has diagnostic or predictive value. For example, a set of antibodies (MMPs/TIMPs) or primers/probes (miRs) specific to each of the biomarkers being examined can be used simultaneously to get a value for each of the biomarkers with one assay. Alternatively, each biomarker can be measured separately and then the value of each can be combined with the other biomarkers to result in biomarker profile.

Because the sample, preferably a bodily fluid, is obtained from a subject at a particular time, the analysis of the different biomarkers can be performed over a plurality of different times. Removing the sample from the subject results in the sample not accumulating or losing any of the biomarkers present in the sample at that time. Of course the handling of the sample over time can lead to degradation of certain biomarkers and thus careful handling and analysis is required.

The biomarkers can be measured or detected in the sample minutes, hours, days, weeks or months apart. The amount of time between detecting each biomarker can differ. For example, the first and second biomarkers can be detected simultaneously and the third biomarker can be detected a day later. A fourth biomarker can be detected hours after the third biomarker. Thus, there may not be a specific amount of time designated between the detection of the different biomarkers.

In some instances, the detection of the biomarkers is performed hours or days after a subject is diagnosed with TAA or risk factors/symptoms associated with TAA. Although many TAAs are asymptomatic, if several known TAA symptoms are identified, the combination of biomarkers may be measured within days of the presence of the symptoms.

The disclosed methods include the determination, identification, indication, correlation, diagnosis, prognosis, etc. (which can be referred to collectively as “identifications”) of subjects, diseases, conditions, states, etc. based on measurements, detections, comparisons, analyses, assays, screenings, etc. For example, levels or amounts of the combination of MMPs, TIMPs, and/or miRs can be used to identify subjects that have or are at risk of cardiovascular diseases or dysfunctions, such as thoracic aortic aneurysm. Such identifications are useful for many reasons. For example, and in particular, such identifications allow specific actions to be taken based on, and relevant to, the particular identification made. For example, diagnosis of a particular disease or condition in particular subjects (and the lack of diagnosis of that disease or condition in other subjects) has the very useful effect of identifying subjects that would benefit from treatment, actions, behaviors, etc. based on the diagnosis. For example, treatment for a particular disease or condition in subjects identified is significantly different from treatment of all subjects without making such an identification (or without regard to the identification). Subjects needing, or that could benefit from, the treatment will receive it and subjects that do not need, or would not benefit from, the treatment will not receive it.

Accordingly, also disclosed herein are methods involving taking particular actions following and based on the disclosed identifications. For example, disclosed are methods involving creating a record of an identification (in physical—such as paper, electronic, or other—form, for example). Thus, for example, creating a record of an identification based on the disclosed methods differs physically and tangibly from merely performing a measurement, detection, comparison, analysis, assay, screen, etc. Such a record is particularly substantial and significant in that it allows the identification to be fixed in a tangible form that can be, for example, communicated to others (such as those who could treat, monitor, follow-up, advise, etc. the subject based on the identification); retained for later use or review; used as data to assess sets of subjects, treatment efficacy, accuracy of identifications based on different measurements, detections, comparisons, analyses, assays, screenings, etc., and the like. For example, such uses of records of identifications can be made, for example, by the same individual or entity as, by a different individual or entity than, or a combination of the same individual or entity as and a different individual or entity than, the individual or entity that made the record of the identification. The disclosed methods of creating a record can be combined with any one or more other methods disclosed herein, and in particular, with any one or more steps of the disclosed methods of identification.

As another example, disclosed are methods including making one or more further identifications based on one or more other identifications. For example, particular treatments, monitorings, follow-ups, advice, etc. can be identified based on the other identification. For example, identification of subject as having a disease or condition with a high level of a particular component can be further identified as a subject that could or should be treated with a therapy based on or directed to the high level component. A record of such further identifications can be created (as described above, for example) and can be used in any suitable way. Such further identifications can be based, for example, directly on the other identifications, a record of such other identifications, or a combination. Such further identifications can be made, for example, by the same individual or entity as, by a different individual or entity than, or a combination of the same individual or entity as and a different individual or entity than, the individual or entity that made the other identifications. The disclosed methods of making a further identification can be combined with any one or more other methods disclosed herein, and in particular, with any one or more steps of the disclosed methods of identification.

As another example, disclosed are methods including treating, monitoring, following-up with, advising, etc. a subject identified in any of the disclosed methods. Also disclosed are methods including treating, monitoring, following-up with, advising, etc. a subject for which a record of an identification from any of the disclosed methods has been made. For example, particular treatments, monitorings, follow-ups, advice, etc. can be used based on an identification and/or based on a record of an identification. For example, a subject identified as having a disease or condition with a high level of a particular component (and/or a subject for which a record has been made of such an identification) can be treated with a therapy based on or directed to the high level component. Such treatments, monitorings, follow-ups, advice, etc. can be based, for example, directly on identifications, a record of such identifications, or a combination. Such treatments, monitorings, follow-ups, advice, etc. can be performed, for example, by the same individual or entity as, by a different individual or entity than, or a combination of the same individual or entity as and a different individual or entity than, the individual or entity that made the identifications and/or record of the identifications. The disclosed methods of treating, monitoring, following-up with, advising, etc. can be combined with any one or more other methods disclosed herein, and in particular, with any one or more steps of the disclosed methods of identification.

The biomarkers can be measured or detected in a variety of ways known in the art. For example, the MMPs and TIMPs can be measured at the nucleic acid or protein level. The detection of miRs can be performed using common nucleic acid identification techniques.

Techniques available for measuring nucleic acid, such as RNA, content are well known in the art and routinely practiced by those in the clinical diagnostics field. Such techniques can include reverse transcription of RNA to produce cDNA and an optional amplification step followed by the detection of the cDNA or a product thereof. Examples of detecting nucleic acids include but are not limited to PCR, reverse-transcription PCR, real-time quantitative PCR (Jiang et al 2003a and 2004. Jiang W G, Watkins G, Lane J, Douglas-Jones A, Cunnick G H, Mokbel M, Mansel R E. Prognostic value of Rho family and and rho-GDIs in breast cancer. Clinical Cancer Research, 2003a, 9, 6432-6440; Jiang W G, Watkins G, Fodstad O, Douglas-Jones A, Mokbel K, Mansel R E. Differential expression of the CCN family members Cyr61 from CTGF and Nov in human breast cancer. Endocrine Related Cancers, 2004, 11: 781-791.), northern blot, southern blot, and dot blots.

Alternatively, determining expression levels can involve assaying for the protein encoded by each of the said biomarkers. Protein assays typically, but not exclusively, involve the use of agents that bind to the relevant proteins. Common protein binding agents are antibodies and, most ideally, monoclonal antibodies which, advantageously, have been labeled with a suitable tag whereby the existence of the bound antibody can be determined. Assay techniques for identifying or detecting proteins are well known to those skilled in the art and are used every day by workers in the field of clinical diagnostics. Such assay techniques can be applied by the skilled worker to utilize the invention. Examples of protein detection assays include, but are not limited to, immunoassays such as enzyme-linked immunosorbant assays (ELISA), western blots, dot blots, radioimmunoassay (RIA), fluoroimmunoassay (FIA), immunoprecipitation and the like.

In some embodiments, the assayed levels of miR, MMP, TIMP, or combination thereof, are used to derive a TAA score that predicts the presence and severity of TAA in a subject. The assayed levels contain numerous data points that are best managed and stored in a computer readable form. Therefore, in preferred embodiments, the TAA score is a regression value derived from the assayed levels as a weighted function of the assayed levels. The weighted function can be derived from linear regression analysis of experimental results comparing assayed levels of normal subjects versus those with TAA. Each level can be multiplied by a weighting constant and summed.

In some embodiments, a TIMP score is determined as an indicator of proteolytic activity that is derived from the ratio of the abundance of a given MMP divided by the sum of TIMP1+2+3+4 abundance. In some embodiments, there is an relationship between TIMP score and aortic size.

The levels may also be analyzed by principal component analysis (PCA) to derive a risk score. PCA is a mathematical procedure that uses an orthogonal transformation to convert a set of observations of possibly correlated variables into a set of values of linearly uncorrelated variables called principal components. The number of principal components is less than or equal to the number of original variables. This transformation is defined in such a way that the first principal component has the largest possible variance (that is, accounts for as much of the variability in the data as possible), and each succeeding component in turn has the highest variance possible under the constraint that it be orthogonal to (i.e., uncorrelated with) the preceding components.

Prior to analysis, the data in each dataset can be collected, usually in duplicate or triplicate or in multiple replicates. The data may be manipulated, for example raw data may be transformed using standard curves, and the average of replicate measurements used to calculate the average and standard deviation for the sample. These values may be transformed before being used in the models, e.g. log-transformed, Box-Cox transformed, etc. This data can then be input into an analytical process with defined parameter. The analytic classification process may be any type of learning algorithm with defined parameters, or in other words, a predictive model. In general, the analytical process will be in the form of a model generated by a statistical analytical method such as those described below. Examples of such analytical processes may include a linear algorithm, a quadratic algorithm, a polynomial algorithm, a decision tree algorithm, or a voting algorithm. Using any suitable learning algorithm, an appropriate reference or training dataset can be used to determine the parameters of the analytical process to be used for classification, i.e., develop a predictive model. The reference or training dataset to be used will depend on the desired classification to be determined. The dataset may include data from two, three, four or more classes. The number of features that may be used by an analytical process to classify a test subject with adequate certainty is 2 or more. In some embodiments, it is 3 or more, 4 or more, 10 or more, or between 10 and 200. Depending on the degree of certainty sought, however, the number of features used in an analytical process can be more or less, but in all cases is at least 2. In one embodiment, the number of features that may be used by an analytical process to classify a test subject is optimized to allow a classification of a test subject with high certainty. Suitable data analysis algorithms are known in the art. In one embodiment, a data analysis algorithm of the disclosure comprises Classification and Regression Tree (CART), Multiple Additive Regression Tree (MART), Prediction Analysis for Microarrays (PAM), or Random Forest analysis. Such algorithms classify complex spectra from biological materials, such as a blood sample, to distinguish subjects as normal or as possessing biomarker levels characteristic of a particular condition (e.g., relapse behavior). In other embodiments, a data analysis algorithm of the disclosure comprises ANOVA and nonparametric equivalents, linear discriminant analysis, logistic regression analysis, nearest neighbor classifier analysis, neural networks, principal component analysis, hierarchical cluster analysis, quadratic discriminant analysis, regression classifiers and support vector machines.

As will be appreciated by those of skill in the art, a number of quantitative criteria can be used to communicate the performance of the comparisons made between a test marker profile and reference marker profiles. These include area under the curve (AUC), hazard ratio (HR), relative risk (RR), reclassification, positive predictive value (PPV), negative predictive value (NPV), accuracy, sensitivity and specificity, Net reclassification Index, Clinical Net reclassification Index. In addition, other constructs such a receiver operator curves (ROC) can be used to evaluate analytical process performance.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a microRNA measurement is disclosed and discussed and a number of modifications that can be made to the method are discussed, each and every combination and permutation of the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

The disclosed methods and compositions are applicable to numerous areas including, but not limited to, diagnose, assess prognosis, monitor improvement or deterioration, or monitor the progress of treatment of thoracic aortic aneurysm. Other uses are disclosed, apparent from the disclosure, and/or will be understood by those in the art.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Plasma Biomarkers for Distinguishing Etiological Subtypes of Thoracic Aortic Aneurysm Disease

Introduction

Thoracic aortic aneurysm (TAA) is an insidious and potentially devastating disease process. Despite advancements in our understanding of the pathobiology of thoracic aortic aneurysms, these advancements have yet to be translated into significant advancements in screening, diagnosis, tracking and treatment of TAAs.

From a biological standpoint, specific proteinases such as the matrix metalloproteinases (MMPs) and their endogenous inhibitors (TIMPs) are implicated in the pathogenesis of ascending thoracic aortic aneurysms (Fedak P W, et al. J Thorac Cardiovasc Surg 2003; 126:797-806; LeMaire S A, et al. J Surg Res 2005; 123:40-8; Ikonomidis J S, et al. Circulation 2006; 114:1365-70; Ikonomidis J S, et al. J Thorac Cardiovasc Surg 2007; 133:1028-36). In addition, specific and different cassettes of MMPs and TIMPs are present in ascending TAAs with different etiologies, such as those associated with congenitally bicuspid aortic valves (BAVs) or tricuspid aortic valves (TAVs) (Fedak P W, et al. J Thorac Cardiovasc Surg 2003; 126:797-806; LeMaire S A, et al. J Surg Res 2005; 123:40-8; Ikonomidis J S, et al. Circulation 2006; 114:1365-70; Ikonomidis J S, et al. J Thorac Cardiovasc Surg 2007; 133:1028-36). In addition, different types of microRNAs (miRs) are expressed within these aneurysms (Jones J A, et al. Circ Cardiovasc Genet 2011; 4:605-13).

Many of these agents can be reliably measured in plasma, providing a potentially valuable strategy to identify and follow the progression of TAAs. Accordingly, the present study sought to identify circulating plasma factors that could distinguish and predict the etiological subtypes of aneurysm disease.

Methods

Patient Demographics. Matched tissue and plasma specimens from 42 patients with ascending aortic aneurysms (n=21 BAV patients, n=21 TAV patients) were taken from the widest region of the ascending aorta at the time of surgical resection or aortic valve replacement. No patients had aortic dissection, inflammatory aortic disease, or known connective tissue disorder. Normal aortic specimens were similarly harvested from the ascending aorta of heart transplant donors or recipients (n=10). Group mean ages were 58±6 years Normal, 59±2 years BAV and 70±2 years TAV (TAV p<0.05 from BAV and Normal). Seventy percent of Normal, 71% of BAV and 52% of TAV patients were male. Aortic diameters were 3.8±0.2 cm Normal, 5.2±0.2 cm BAV and 5.7±0.2 cm TAV (TAV, BAV p<0.05 from Normal). Normal aortic tissue and plasma specimens were snap frozen and stored at −80° C. until analyzed. This study was approved by the institutional review boards of the Medical University of South Carolina, Duke University, and the University of Pennsylvania. Informed consent was obtained from all patients.

Tissue Samples. For each tissue sample, 5 mg of frozen tissue was weighed and homogenized using a bead-mill homogenizer (Qiagen, Valencia, Calif.). Total RNA was extracted from tissue homogenates (mirVana PARIS miRNA Isolation kit; Applied Biosystems/Ambion Austin, Tex.) and analyzed for RNA quality and quantity using an Experion Automated Electrophoresis System (RNA StdSens Analysis Kit, Bio-Rad Laboratories, Hercules, Calif.). Ten ng of total RNA was reversed transcribed (TaqMan MicroRNA Reverse Transcription Kit; Applied Biosystems) for each miR of interest, and quantitative PCR was performed. Each tissue sample was analyzed for the following miRs: hsa-miR-1, hsa-miR-21, hsa-miR-29a, has-miR-133a, hsa-miR-143, and hsa-miR-145.

Plasma Samples. RNA was isolated from 50 μl of plasma (mirVana PARIS Protein and RNA isolation System for Small RNAs; Ambion, AM1556) following the manufacturer's instructions. The isolated RNA (40 μl) was then incubated for one hour at room temperature with 1.3 units of Heparinase-I (IBEX Pharmaceuticals Inc., PN 50-010-001) in a buffer containing 20 mM Tris, pH 7.5, 50 mM NaCl, 4 mM CaCl₂ and 0.01% BSA. Five μl of treated RNA was reverse transcribed for each miR of interest and quantitative PCR was performed. Each plasma sample was analyzed for the following miRs: hsa-miR-1, hsa-miR-21, hsa-miR-29a, has-miR-133a, hsa-miR-143, and hsa-miR-145.

Quantitative Polymerase Chain Reaction (QPCR). For both tissue and plasma samples, the reverse transcription product was amplified with gene specific TaqMan® primer/probe sets using the TaqMan® Universal PCR Master Mix with no AmperErase UNG (Cat #4324020, Applied Biosystems, Carlsbad, Calif.) in a CFX96 Real-Time PCR Detection System (Bio-Rad, Hercules, Calif.). The thermal cycling protocol was conducted as follows: 10 minutes at 95° C., followed by 40 cycles of 95° C. for 15 seconds, and 60° C. for 1 minute. Negative PCR controls were run to verify the absence of genomic DNA contamination (no reverse transcription control). Fluorescence was recorded at regular intervals following the 60° C. annealing/extension segment of the PCR reaction and real-time data showing relative fluorescence versus cycle number was analyzed. Because of the paucity of good internal PCR controls for plasma specimens, miR expression in both tissue and plasma (for consistency of measurement) was determined from a Ct value (expression=2^{(-ΔCt)}) where ΔCt was derived for each individual specimen, and calculated by subtracting the mean Ct value for all targets measured from the individual Ct value of a given PCR target, as previously described.⁶⁻⁷ Results were then reported as a mean±SEM for each miR measured in either tissue or plasma.

MMP/TIMP Multiplex Suspension Array (MSA). For the tissue specimens, thawed tissue was transferred to a cold buffer (volume 1:6 w/v) containing 10 mM cacodylic acid pH 5.0, 0.15 M NaCl, 10 mM ZnCl2, 1.5 mM NaN₃, and 0.01% Triton X-100 (v/v), and homogenized using a bead-mill homogenizer (Qiagen, Valencia, Calif.). The homogenates were then centrifuged (800×g, 10 min, 4° C.), and 20 μg was analyzed using an MSA approach. The following MMPs (-1, -2, -3, -7, -8, -9, -12, and -13) and TIMPs (-1, -2, -3, and -4) were examined as previously described (Ikonomidis J S, et al. Ann Thorac Surg 2012 93:457-63). The plasma specimens were analyzed in a similar fashion following dilution (1:100 for MMPs-2, and -9; 1:10 for the MMPs-1,-3,-7-8,-12, and -13; and 1:20 for the TIMPs), as previously described (Essa E M, et al. J Card Fail 2012 18:487-92). In both cases, samples were incubated on a microplate shaker (room temperature, 2 hours), filtered, and washed 3 times with 100 μl of wash buffer. Diluted goat anti-human polyclonal biotinylated antibodies (50 μl, analyte specific [included with antibody-conjugated bead kits], R&D Systems) were then added to each well and the specimens were again incubated on a microplate shaker (room temperature, 1 hour). The beads were filtered and washed as before, and streptavidin-phycoerythrin (50 μl, R&D Systems) was added to each well for 30 minutes at room temperature. After a final filtration and wash, the beads were analyzed using the Bio-Plex System; fluorescence was measured and then compared with standard curves for each analyte also run on the same plate. Protein quantities were calculated using Bio-Plex Manager Software 4.1 and expressed as absolute concentration in pg/ml.

Data Analysis. Expression levels of miRs and protein abundance of the MMPs/TIMPs were analyzed in two ways. First, all QPCR and MSA results were subjected to a Shapiro-Wilk test for normality. For the unequally distributed analytes, the absolute values were log transformed. Then all values were subjected to a one-way analysis of variance (prcomp module, Stata) with Tukey's wholly significant difference post-hoc analysis for separation of means to determine differences between the referent controls, BAV, and TAV groups. Second, the percent change of miR and MMP/TIMP levels in the BAV and TAV groups were computed and compared to the referent controls using a one-sample mean comparison test with the hypothesized mean set at 100%. Analysis of variance with Tukey's wholly significant difference post-hoc analysis (prcomp module) was used to determined differences between BAV and TAV groups. Linear regression analysis was performed to identify significant relationships between tissue and plasma levels of each analyte. Additionally, plasma biomarkers were assessed for univariate association with the presence of aortic aneurysm using logistic regression models. Receiver operating characteristic curves was then generated to compute an area under the curve (AUC) for each individual biomarker. Those biomarkers with p values of less than 0.25 were considered for inclusion in a multiple logistic regression model. Using forward stepwise variable selection, biomarkers were added to the model with the variable most strongly associated with outcome (presence of a TAA) being added to the model until no more variables met the entry criterion of α<=0.20. The α-level was set at 0.20 to ensure that even marginally predictive biomarkers were captured. Logistic regression analysis was performed to determine the coefficients and intercepts for biomarkers that were found to be significant predictors of aneurysm development in multivariable analysis. Discrimination and classification of the fitted multivariable models were assessed by using the generated equation and computing the corresponding sensitivity, specificity, positive predictive, and negative predictive values (Altman D G, Bland J M. Diagnostic tests 2: Predictive values. BMJ 1994; 309:102). Finally, relative proteolytic balance was expressed as the ratio of MMP abundance to a composite TIMP score composed of the sum of TIMP-1, TIMP-2, TIMP-3, and TIMP-4 abundance in each sample. Changes in the ratio of MMP abundance to a composite TIMP score were determined by using a one-sample mean comparison test with the hypothesized mean for the referent controls set at 100%. All statistical calculations were made using the Stata software package (v8.2; StataCorp LP, College Station, Tex.). In all cases, p<0.05 was considered significant.

Results

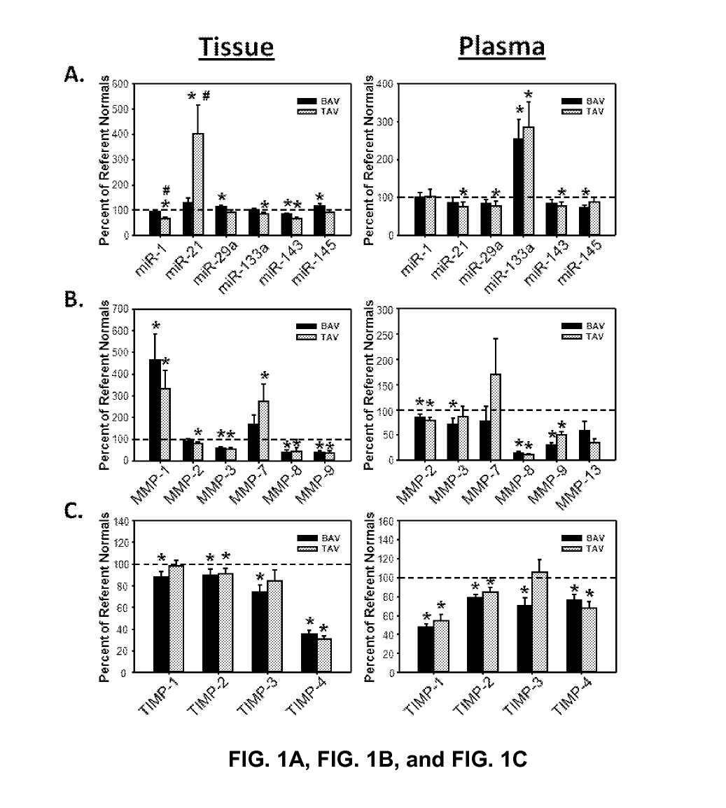

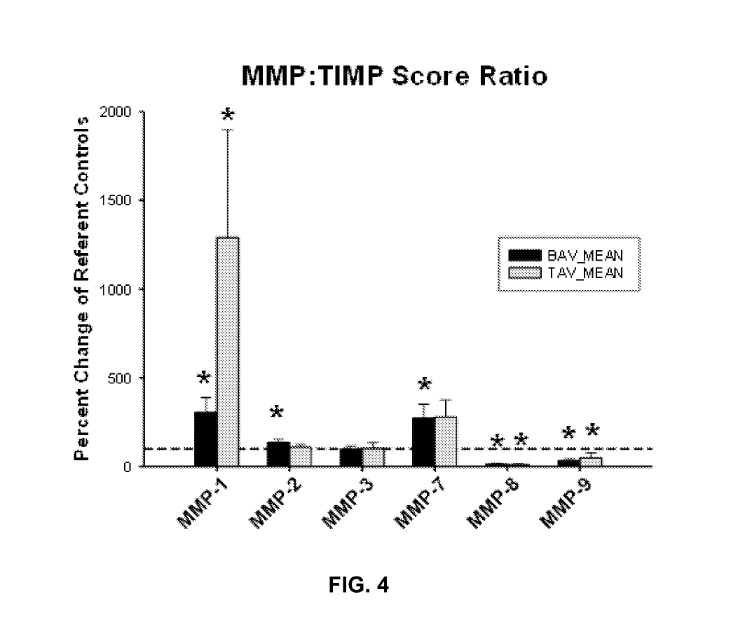

Tissue and plasma measurements of MMPs, TIMPs and miRs standardized to normal aorta or normal plasma are shown in FIG. 1. Absolute value measurements are summarized in Table 1 (Tissue) and Table 2 (Plasma). There were significant differences in the tissue and/or plasma levels of several analytes with respect to control values. Moreover, a differential expression of certain analytes was observed in TAAs from the BAV and TAV groups. For example, tissue levels of miR-1 and miR-21 were differentially altered in the TAV group compared to the BAV group (Table 1). Lastly, relative proteolytic balance in the tissue specimens was expressed as the ratio of MMP abundance to a composite TIMP score composed of the sum of TIMP-1, TIMP-2, TIMP-3, and TIMP-4 abundance in each sample, shown in the FIG. 4. Relative to normal aorta, BAV proteolytic balance was significantly increased for MMP-1, -2 and-7, and decreased for MMP-8 and-9. In contrast, TAV proteolytic balance relative to normal aorta was significantly increased only for MMP-1 and decreased for MMP-8 and -9.

All analytes were subjected to a Shapiro-Wilk test for normality. For the unequally distributed analytes, the absolute values were log transformed and subjected to a one-way analysis of variance (prcompw module) with Tukey's wholly significant difference post-hoc analysis for separation of means to determine differences between the referent controls, BAV, and TAV groups.

TABLE 1
Absolute values for miR expression (no unit) and protein abundance
of MMPs and TIMPs (pg/ml) in aortic tissue from normal and
TAA patients with BAV or TAV.

| | | | |
|---|---|---|---|
| Tissue Analyte | Control | BAV | TAV |

microRNA (2^{-ΔCt})

| | | | |
|---|---|---|---|
| miR-1 (1 × 10⁻³) | 57.8 ± 2.4 | 54.3 ± 3.3 | 39.4 ± 3.3*^{#} |
| miR-21 (1 × 10⁻²) | 169.7 ± 21.4 | 217.7 ± 32.3 | 683.0 ± 191.0*^{#} |
| miR-29a (1 × 10⁻²) | 242.4 ± 33.3 | 274.7 ± 17.2 | 220.2 ± 25.8 |
| miR-133a (1 × 10⁻³) | 80.3 ± 5.2 | 81.7 ± 5.8 | 69.2 ± 5.1 |
| miR-143 (1 × 10⁻²) | 830.9 ± 37.9 | 703.7 ± 45.9 | 560.6 ± 49.1* |
| miR-145 (1 × 10⁻²) | 771.0 ± 37.9 | 910.8 ± 72.1 | 715.0 ± 75.6 |

MMPs (pg/mL)

| | | | |
|---|---|---|---|
| MMP-1 | 3.4 ± 1.0 | 15.7 ± 4.0 | 11.3 ± 2.8 |
| MMP-2 (1 × 10²) | 71.6 ± 13.0 | 64.1 ± 7.7 | 56.9 ± 5.4 |
| MMP-3 | 292.9 ± 45.5 | 165.2 ± 22.4 | 160.6 ± 20.2 |
| MMP-7 (1 × 10¹) | 8.8 ± 2.9 | 14.6 ± 3.8 | 24.0 ± 7.2 |
| MMP-8 (1 × 10¹) | 294.1 ± 128.8 | 121.7 ± 29.3 | 128.5 ± 49.8 |
| MMP-9 (1 × 10²) | 74.9 ± 49.6 | 28.3 ± 7.1 | 26.0 ± 7.9 |
| MMP-12 | ND | ND | ND |
| MMP-13 | ND | ND | ND |

TIMPs (pg/mL)

| | | | |
|---|---|---|---|
| TIMP-1 (1 × 10²) | 113.6 ± 13.7 | 100.2 ± 5.9 | 111.3 ± 6.1 |
| TIMP-2 (1 × 10²) | 127.9 ± 17.1 | 114.2 ± 7.5 | 116.3 ± 6.5 |
| TIMP-3 (1 × 10²) | 22.2 ± 3.0 | 16.5 ± 1.5 | 18.8 ± 2.2 |
| TIMP-4 | 141.9 ± 33.7 | 49.8 ± 5.7* | 43.6 ± 4.6* |
| Sample Size (n) | 10 | 21 | 21 |

*p < 0.05 vs. Control
^{#}p < 0.05 vs. BAV

TABLE 2
Absolute values for miR expression (no unit) and protein abundance
of MMPs and TIMPs (pg/ml) in plasma from patients with normal
aorta and TAA patients with BAV or TAV.

| | | | |
|---|---|---|---|
| Plasma Analyte | Control | BAV | TAV |

microRNA (2^{-ΔCt})

| | | | |
|---|---|---|---|
| miR-1 (1 × 10⁻³) | 70.1 ± 15.7 | 70.8 ± 8.2 | 72.7 ± 13.0 |
| miR-21 | 42.5 ± 11.7 | 36.7 ± 5.8 | 32.4 ± 5.6 |
| miR-29a | 7.2 ± 1.4 | 6.1 ± 0.7 | 5.7 ± 0.8 |
| miR-133a (1 × 10⁻³) | 45.0 ± 12.7 | 114.5 ± 22.9 | 128.0 ± 30.0 |
| miR-143 (1 × 10⁻¹) | 14.4 ± 2.5 | 12.2 ± 1.4 | 11.3 ± 1.4 |
| miR-145 (1 × 10⁻²) | 94.3 ± 11.4 | 69.8 ± 5.0 | 84.3 ± 11.0 |

MMPs (pg/mL)

| | | | |
|---|---|---|---|
| MMP-1 | ND | ND | ND |
| MMP-2 (1 × 10⁴) | 53.4 ± 4.7 | 45.6 ± 3.6 | 41.9 ± 3.1 |
| MMP-3 (1 × 10³) | 22.8 ± 3.6 | 16.1 ± 2.9 | 19.7 ± 4.6 |
| MMP-7 (1 × 10²) | 20.8 ± 18.4 | 16.0 ± 6.3 | 35.5 ± 14.5 |
| MMP-8 (1 × 10²) | 91.0 ± 28.9 | 13.3 ± 3.5* | 10.8 ± 1.3* |
| MMP-9 (1 × 10⁴) | 83.6 ± 21.5 | 25.6 ± 3.5* | 41.8 ± 5.3*^{#} |
| MMP-12 | ND | ND | ND |
| MMP-13 (1 × 10²) | 24.9 ± 15.0 | 11.0 ± 3.6 | 8.5 ± 3.6 |

TIMPs (pg/mL)

| | | | |
|---|---|---|---|
| TIMP-1 (1 × 10³) | 116.4 ± 24.7 | 56.0 ± 3.2* | 63.8 ± 7.2* |
| TIMP-2 (1 × 10³) | 55.2 ± 2.8 | 43.6 ± 1.7* | 46.9 ± 2.4 |
| TIMP-3 (1 × 10²) | 34.2 ± 4.6 | 24.1 ± 2.8 | 36.2 ± 4.6 |
| TIMP-4 (1 × 10²) | 19.4 ± 2.9 | 14.7 ± 1.2 | 13.3 ± 1.1 |
| Sample Size (n) | 10 | 21 | 21 |

*p < 0.05 vs. Control
^{#}p < 0.05 vs. BAV

TABLE 3
Area-under-the-curve (AUC) for individual plasma analytes

| | | |
|---|---|---|
| All Aneurysms | BAV | TAV |

microRNA

| | | | |
|---|---|---|---|
| miR-1 | 0.4571 (0.84) | 0.4795 (0.97) | 0.4306 (0.75) |
| miR-21 | 0.4538 (0.46) | 0.4850 (0.65) | 0.4211 (0.37) |
| miR-29a | 0.4077 (0.31) | 0.4300 (0.41) | 0.3842 (0.35) |
| miR-133a | 0.7654 (<0.01) | 0.7602 (<0.01) | 0.7712 (<0.01) |
| miR-143 | 0.3596 (0.23) | 0.3567 (0.36) | 0.3626 (0.21) |
| miR-145 | 0.3385 (0.21) | 0.2700 (0.02) | 0.4105 (0.65) |

MMPs

| | | | |
|---|---|---|---|
| MMP-1 | ND | ND | ND |
| MMP-2 | 0.2615 (0.03) | 0.3050 (0.07) | 0.2158 (0.02) |
| MMP-3 | 0.3231 (0.45) | 0.3350 (0.28) | 0.3105 (0.62) |
| MMP-7 | 0.6071 (0.80) | 0.5769 (0.66) | 0.6333 (0.56) |
| MMP-8 | 0.1321 (<0.01) | 0.1275 (<0.01) | 0.1368 (<0.01) |
| MMP-9 | 0.1667 (<0.01) | 0.0950 (<0.01) | 0.2421 (<0.01) |
| MMP-12 | ND | ND | ND |
| MMP-13 | 0.3590 (0.08) | 0.3889 (0.20) | 0.3333 (0.10) |

TIMPs

| | | | |
|---|---|---|---|
| TIMP-1 | 0.2231 (<0.01) | 0.1850 (<0.01) | 0.2632 (<0.01) |
| TIMP-2 | 0.2051 (<0.01) | 0.1300 (<0.01) | 0.2842 (0.03) |
| TIMP-3 | 0.3833 (0.44) | 0.2575 (0.04) | 0.5158 (0.75) |
| TIMP-4 | 0.3205 (0.04) | 0.3650 (0.18) | 0.2737 (0.01) |

Values presented as: AUC (p-value) for each individual analyte
Bolded cells indicate AUC values that are statistically significant in univariate analysis

TABLE 4
Percent change of referent control for miR expression (no unit)
and protein abundance of MMPs and TIMPs (pg/ml) in aortic
tissue from TAA patients with BAV or TAV

| | | | |
|---|---|---|---|
| Tissue Analyte | Control | BAV | TAV |

microRNA (%)

| | | | |
|---|---|---|---|
| miR-1 | 100 ± 4 | 94 ± 6 | 68 ± 6* |
| miR-21 | 100 ± 13 | 128 ± 19 | 403 ± 113* |
| miR-29a | 100 ± 14 | 113 ± 7* | 91 ± 11 |
| miR-133a | 100 ± 6 | 102 ± 7 | 86 ± 6* |
| miR-143 | 100 ± 12 | 85 ± 6 | 67 ± 6* |
| miR-145 | 100 ± 5 | 118 ± 9* | 93 ± 10 |

MMPs (pg/mL)

| | | | |
|---|---|---|---|
| MMP-1 | 100 ± 28 | 464 ± 121* | 332 ± 82* |
| MMP-2 | 100 ± 18 | 90 ± 11 | 79 ± 8* |
| MMP-3 | 100 ± 16 | 56 ± 7* | 55 ± 7* |
| MMP-7 | 100 ± 33 | 167 ± 43 | 274 ± 82* |
| MMP-8 | 100 ± 44 | 41 ± 9 | 44 ± 10* |
| MMP-9 | 100 ± 66 | 38 ± 10* | 35 ± 11* |
| MMP-12 | ND | ND | ND |
| MMP-13 | ND | ND | ND |

TIMPs (pg/mL)

| | | | |
|---|---|---|---|
| TIMP-1 | 100 ± 12 | 88 ± 5* | 95 ± 5 |
| TIMP-2 | 100 ± 13 | 89 ± 6* | 91 ± 5* |
| TIMP-3 | 100 ± 14 | 74 ± 7* | 85 ± 10 |
| TIMP-4 | 100 ± 24 | 35 ± 4* | 31 ± 3* |
| Sample Size (n) | 10 | 21 | 21 |

*p < 0.05 vs. Control
All analytes were calculated as percent change of referent control group which was set at 100%.
A one-sided one sample t-test was used to detect significant difference from the referent control group.

TABLE 5
Percent change of referent control for miR expression and protein
abundance of MMPs and TIMPs in plasma from TAA patients
with BAV or TAV

| | | | |
|---|---|---|---|
| Plasma Analyte | Control | BAV | TAV |

microRNA (%)

| | | | |
|---|---|---|---|
| miR-1 | 100 ± 4 | 101 ± 12 | 104 ± 19 |
| miR-21 | 100 ± 13 | 86 ± 14 | 76 ± 113* |
| miR-29a | 100 ± 14 | 84 ± 10 | 79 ± 11* |
| miR-133a | 100 ± 6 | 245 ± 51* | 284 ± 67* |
| miR-143 | 100 ± 12 | 84 ± 10 | 78 ± 10* |
| miR-145 | 100 ± 5 | 74 ± 5* | 89 ± 12 |

MMPs (pg/mL)

| | | | |
|---|---|---|---|
| MMP-1 | ND | ND | ND |
| MMP-2 | 100 ± 9 | 85 ± 7* | 79 ± 6* |
| MMP-3 | 100 ± 16 | 70 ± 13* | 86 ± 20 |
| MMP-7 | 100 ± 88 | 77 ± 30 | 170 ± 70 |
| MMP-8 | 100 ± 32 | 15 ± 4* | 11 ± 1* |
| MMP-9 | 100 ± 26 | 31 ± 4* | 50 ± 6* |
| MMP-12 | ND | ND | ND |
| MMP-13 | 100 ± 60(n = 3) | 58 ± 19* (n = 7) | 34 ± 9* (n = 7) |

TIMPs (pg/mL)

| | | | |
|---|---|---|---|
| TIMP-1 | 100 ± 21 | 48 ± 3* | 55 ± 6* |
| TIMP-2 | 100 ± 5 | 79 ± 3* | 85 ± 4* |
| TIMP-3 | 100 ± 13 | 70 ± 8* | 105 ± 14 |
| TIMP-4 | 100 ± 15 | 76 ± 6* | 68 ± 6* |
| Sample Size (n) | 10 | 21 | 21 |

*p < 0.05 vs. Control
All analytes were calculated as percent change of referent control group which was set at 100%.
A one-sided one sample t-test was used to detect significant difference from the referent control group.

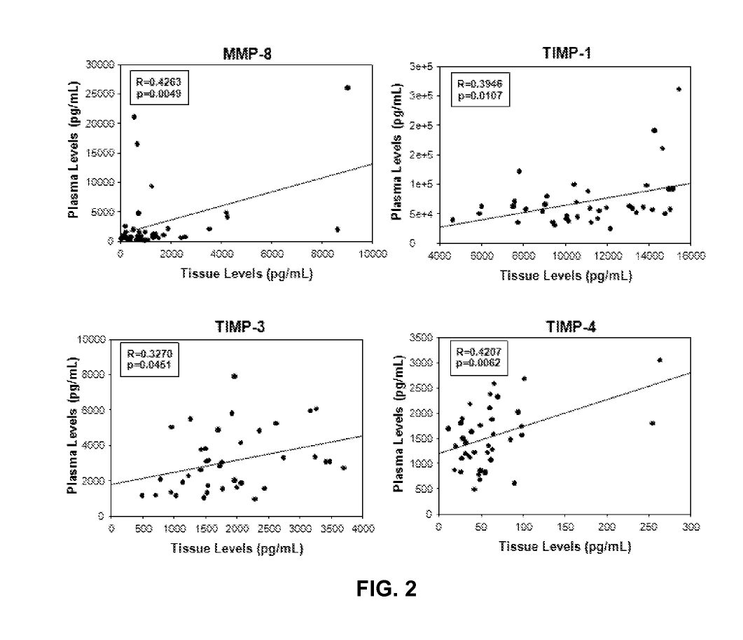

Linear regression analysis, performed to identify significant relationships between tissue and plasma levels of each analyte, revealed significant linear relationships only for MMP-8 and TIMPs-1,-3 and-4. These results are summarized in FIG. 2.

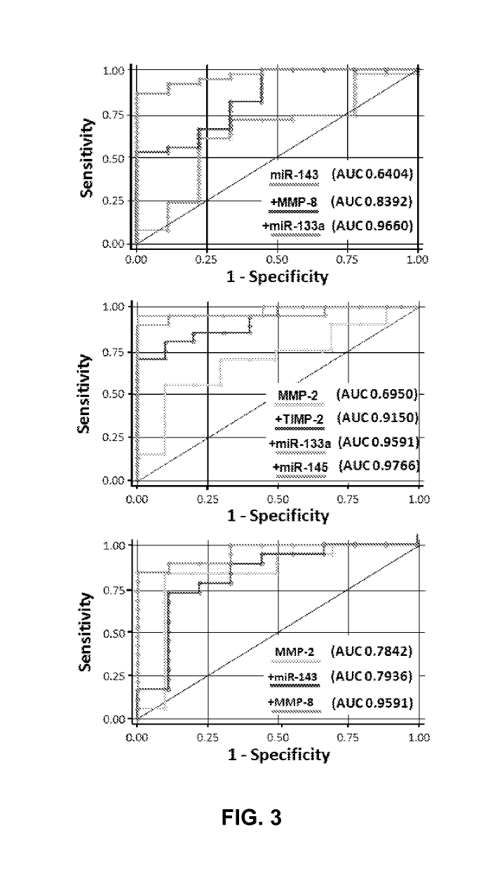

Receiver operator characteristic curve analysis was performed to determine whether plasma levels of the analytes could serve as biomarker(s) for the presence/absence of TAA disease. The AUC values from the univariate analysis are summarized in Table 3. Following this, forward stepwise multivariable receiver operating characteristics analysis was performed, which revealed specific cassettes of analytes predictive of TAA disease, as depicted in FIG. 3. For TAA disease overall, the combination of miR-143, MMP-8 and miR-133a maximized AUC values to 0.9660. For TAAs associated with BAV, the combination of MMP-2, TIMP-2, miR-143, miR-133a and miR-145 maximized AUC values to 0.9766. For TAAs associated with TAV, the combination of MMP-2, miR-143 and MMP-8 maximized AUC values to 0.9591.

Logistic regression coefficients and intercepts for biomarkers that were found to be significant predictors of aneurysm development in multivariate analysis were computed.

For all TAA, the equation: Prob(control/aneurysm)=(2.771357×miR143)−(0.0008061×MMP-8)+(50.81172×miR133a)−3.615998; (r²=0.59, p<0.001); yielded a positive predictive value of 0.92, and negative predictive value of 0.58 a sensitivity of 0.87 and a specificity of 0.70.

For the BAV group, the equation: Prob(control/aneurysm)=(−0.0000152×MMP-2)−(0.0003459×TIMP-2)+(25.91564×miR133a)−(8.569773×miR145)+29.68998; (r²:0.73, p<0.001); resulted a positive predictive value of 0.95, a negative predictive value of 1.00, a sensitivity of 0.95 and a specificity of 1.00.

For the TAV group, the equation: Prob(control/aneurysm)=((−0.0000284×MMP-2)+(4.211116×miR143)−(0.0020155−MMP-8)+14.55217; (r²:0.67, p<0.001); yielded a positive predictive value of 0.89, a negative predictive value of 0.80 a sensitivity of 0.89 and a specificity of 0.80.

Overall, unique tissue and plasma profiles were identified for each TAA etiology (Table 6). MMP-1 was increased in BAV plasma, while it was decreased in TAV plasma. MMP-3 did not change in BAV plasma, but deceased in TAV plasma. TIMP-3 decreased in BAV plasma and did not change in TAV plasma. MicroRNA-133a decreased in BAV plasma, and did not change in TAV plasma, while microRNA-29a did not change in BAV plasma, and decreased in TAV plasma. Together, the unique plasma signature for BAV patients would include increased MMP-1, decreased TIMP-3, and decreased microRNA-133a, while the unique plasma signature for TAV patients would include decreased MMP-3, and decreased microRNA-29a, respectively when compared to plasma from referent control patients without aortic disease.

Ascending TAA tissue and plasma specimens were obtained from BAV (n=29) and TAV (n=24) patients at the time of surgical resection. The protein abundance of key MMPs (-1, -2, -3, -8, -9) and TIMPs (-1, -2, -3, -4), and microRNAs (-1, -21, -29a, -133a, -143, -145) was examined using a multi-analyte protein profiling system or by quantitative PCR, respectively. Results were compared to normal aortic tissue and plasma obtained from patients without aortic disease (n=9).

TABLE 6
Summary of significant plasma and tissue changes.

| | BAV | | TAV | |
|---|---|---|---|---|
| Analyte | Tissue | Plasma | Tissue | Plasma |

MMPs

| | | | | |
|---|---|---|---|---|
| MMP-1 | [symbol] | ↑ | ↑ | ↓ |
| MMP-2 | [symbol] | ↓ | ↓ | ↓ |
| MMP-3 | ↓ | [symbol] | [symbol] | ↓ |
| MMP-8 | ↓ | ↓ | [symbol] | ↓ |
| MMP-9 | ↓ | ↓ | ↓ | ↓ |

TIMPs

| | | | | |
|---|---|---|---|---|
| TIMP-1 | ↓ | ↓ | [symbol] | ↓ |
| TIMP-2 | [symbol] | ↓ | ↓ | ↓ |
| TIMP-3 | [symbol] | ↓ | [symbol] | [symbol] |
| TIMP-4 | ↓ | ↓ | ↓ | ↓ |

microRNAs

| | | | | |
|---|---|---|---|---|
| miR-1 | [symbol] | ND | [symbol] | ND |
| miR-21 | [symbol] | [symbol] | ↑ | [symbol] |
| miR-29a | [symbol] | [symbol] | [symbol] | ↓ |
| miR-133a | [symbol] | ↓ | [symbol] | [symbol] |
| miR-143 | ↓ | [symbol] | ↓ | [symbol] |
| miR-145 | [symbol] | [symbol] | [symbol] | [symbol] |

Plasma Signature
BAV = ↑ MMP-1, ↓ TIMP-3, ↓ microRNA-133a
TAV = ↓ MMP-3, ↓ microRNA-29a

Discussion

The knowledge of the pathobiology of TAAs continues to expand and as such, it is becoming more apparent that this information may be used to improve the diagnosis, tracking, and treatment of these serious conditions. Of particular significance is that TAAs of different etiologic subtypes display different biological patterns which may allow for personalized health care strategies. Currently, TAAs are diagnosed serendipitously during routine physical examinations or assessments for other disease conditions. A screening test for TAAs would be very valuable to identify those individuals who have asymptomatic but potentially life threatening aneurysms, necessitating knowledge of plasma biomarker predictors. As such, this study undertook the task of identifying plasma signatures which could be indicative of specific subtypes of ascending aortic aneurysm disease. This study demonstrated that it was possible to measure a variety of different analytes directly relevant to TAA disease in plasma. Second, very little concordance between plasma measurements and aortic tissue measurements of MMPs, TIMPs, and a specific cassette of miRs was observed. Third, it was shown that aneurysms associated with either bicuspid or tricuspid valves displayed different cassettes of tissue and plasma analytes. Finally, it was demonstrated that it was possible to predict with a high degree of specificity and sensitivity the presence of either aneurysm disease in general or specific etiologic subtypes of aneurysm disease in particular using a plasma multi-analyte regression strategy. These data can be configured to a simple plasma measurement that could aid with the screening of patients and to use these signatures to predict aneurysm activity or changes in aneurysm size.

In the present study, a significant number of MMPs, TIMPs, and miRs were measured in aortic tissue. The results were consistent to some extent with findings that have made before with regards to differential expression of these analytes for aneurysms of different etiologies (Ikonomidis J S, et al. Circulation 2006; 114:1365-70; Ikonomidis J S, et al. J Thorac Cardiovasc Surg 2007; 133:1028-36; Fedak P W, et al. J Thorac Cardiovasc Surg 2003; 126:797-806; LeMaire S A, et al. J Surg Res 2005; 123:40-8). This data further supports the concept that different etiologic subtypes of TAAs display measurable biological differences that can be used to distinguish meaningfully between these disease processes.

It was that a broad range of analytes could be measured in the plasma and that it was possible to generate a different cassette of specific analyte profiles for TAAs associated with bicuspid or tricuspid aortic valves. What was interesting with this study was that in general it was not possible to demonstrate a specific correlation between most tissue and plasma analyte levels. The reasons for this are multifactorial and include the fact that many of the biomarkers measured are primarily intracellular molecules and thus it is difficult to predict how much measurable spillage into plasma would be observed. Furthermore, the half life of the analytes is variable, making it difficult to correlate plasma and tissue concentrations. Also, tissue and plasma storage and handling carries a significant impact on the ability to accurately measure analytes. Although handling of tissue and plasma was an important and rigorous procedure in the laboratory, it is possible that aberrancies in the storage and processing may have affected the results and decreased the degree of concordance between tissue and plasma levels.

An important finding in the study is that step-wise combination of multiple analytes produces an algorithm which is highly sensitive and specific. Hence, aneurysms of different etiologies could have specific plasma regression equations containing a composite of analytes that would accurately predict the presence of disease as a screening tool in patients prior to referral for confirmatory imaging.

The results of this study indicate that specific plasma biosignatures can be generated for aneurysms of different subtypes. These data hold significant importance with regards to the potential advancement of diagnosis, tracking and treatment of thoracic aortic aneurysm disease. Taken together these unique data demonstrate differential plasma profiles of MMPs, TIMPs, and microRNAs in ascending TAA specimens from patients with BAV versus TAV aneurysms. These results indicate that circulating biomarkers can be useful in personalized medicine strategies to distinguish between etiological subtypes of TAAs in patients with aneurysm disease.

### Example 2

### Identification of microRNA Expression Profiles in Bicuspid Aortic Valve Patients with Thoracic Aortic Aneurysms by Next Generation Sequencing

Background

The bicuspid aortic valve (BAV) is a congenital cardiac malformation occurring in 1-2% of the population. BAV patients often develop aortic valve stenosis and regurgitation, and are prone to develop ascending thoracic aortic aneurysms (TAA). The underlying mechanisms that predispose these patients to TAA formation remain unknown. It is now accepted that TAA development, secondary to BAV, is associated with remodeling of the aortic wall and dysregulation in upstream signaling pathways, such as TGFβ. MicroRNAs (miRs) function to regulate protein abundance and specifically target>60% of all mRNA transcripts. Accordingly, this study tested whether a unique pattern of miR expression occurs in the setting of BAV, which is differentially altered in BAV patients that develop TAAs.

Methods/Results

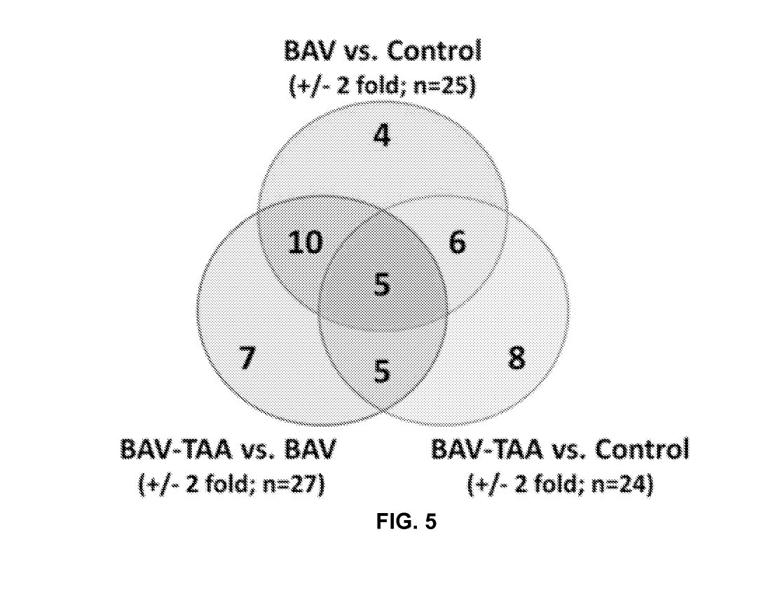

Total RNA was harvested from fresh aortic tissue specimens obtained from non-aneurysmal patients with no valve defects (Control, n=5), BAV patients without TAA (BAV, n=3), and BAV patients with TAA (BAV-TAA, n=4). RNA specimens were subjected to next generation sequencing (Illumina platform), and miR expression was quantitated in each group. Results revealed 561 miRs that were detected and sequenced across all groups. Of these miRs, 25 were differentially expressed (increased or decreased >2-fold) between Control and BAV, 24 between Control and BAV-TAA, and 27 between BAV and BAV-TAA (FIG. 5, top). A bioinformatics approach was taken to identify putative target proteins (TargetScan Human/miRDB). Target pathway analysis (DIANA miRPath) identified four miRs previously not associated with BAV and/or BAV-TAA (miR-128-1, 140, 142, 345), with significantly altered expression, that may regulate TGFβ signaling pathway components (FIG. 5, bottom).

These findings indicate that dysregulated protein abundance, secondary to changes in miR expression, contribute to alterations in TGFβ signaling and TAA development in BAV patients.

TABLE 7

| | BAV vs. | BAV-TAA | BAV-TAA | Putative miR Targets |
|---|---|---|---|---|
| | Control | vs. Control | vs. BAV | in the TGFβ Pathway |
| miR-142 | −2.59* | −1.96* | +1.32 | TAB1, TAB2, TGFBR1, |
| | | | | TBRG1, BMPR2, |
| | | | | BMPR1A, SMAD5, |
| | | | | ACVR1C, LTBP1 |
| miR-345 | +1.08 | −3.89* | −4.20* | SMAD1 |
| miR-140 | −1.06 | +1.89* | +2.00* | ACVR2B, TAB2, |
| | | | | HDAC4, TGFBR1, |
| | | | | BMP2 |
| miR-128-1 | +1.70 | +3.57* | +2.10* | SMURF2, SMAD2, |
| | (p = 0.06) | | | SMAD5, SMAD9, |
| | | | | TGFBR1, TGFBR2, |
| | | | | ACVR2A, SP1 |

### Example 3

### MicroArray Analysis

Table 8 shows microarray results of microRNA that are increased (↑) or decreased (↓) at least 2.5 fold in BAV Aorta (no TAA) vs. Normal Aorta, BAV Aorta (+TAA) vs. Normal Aorta, or BAV Aorta (+TAA) vs. BAV Aorta (no TAA).

TABLE 8
MicroArray Data

| microRNA | Expression Change | Patient Group |
|---|---|---|
| miR-10b | ↑ | |
| miR-133a-1 | ↑ | |
| miR-145 | ↑ | |
| miR-181b-2 | ↑ | |
| miR-30c-2 | ↑ | BAV Aorta |
| miR-30e | ↑ | (no TAA) vs. |
| miR-3676 | ↑ | Normal Aorta |
| miR-125b-2 | ↑ | |
| miR-100 | ↑ | |
| miR-199a-2 | ↑ | |
| miR-23b | ↑ | |
| miR-423 | ↑ | |
| Let-7a-3 | ↓ | |
| miR-22 | ↓ | |
| Let-7f-2 | ↓ | |
| miR-1248 | ↓ | |
| miR-148b | ↓ | |
| miR-181b-1 | ↓ | |
| miR-181c | ↓ | |
| miR-26a-2 | ↓ | |
| miR-30a | ↓ | |
| miR-3607 | ↓ | |
| miR-365b | ↓ | |
| miR-660 | ↓ | |
| miR-146b | ↓ | |
| miR-181b-2 | ↑ | |
| miR-30c-2 | ↑ | |
| miR-30e | ↑ | |
| miR-3676 | ↑ | |
| Let-7f-1 | ↑ | |
| Let-7i | ↑ | |
| miR-127 | ↑ | |
| miR-1307 | ↑ | |
| miR-140 | ↑ | BAV Aorta |
| miR-101-2 | ↑ | (+TAA) vs. |
| miR-3615 | ↑ | Normal Aorta |
| miR-3651 | ↑ | |
| miR-3913-1 | ↑ | |
| miR-501 | ↑ | |
| miR-99b | ↑ | |
| miR-100 | ↑ | |
| miR-146b | ↑ | |
| miR-23b | ↑ | |
| Let-7f-2 | ↓ | |
| miR-1248 | ↓ | |
| Let-7c | ↓ | |
| miR-101-1 | ↓ | |
| miR-199a-2 | ↓ | |
| miR-423 | ↓ | |
| Let-7a-3 | ↑ | |
| miR-148b | ↑ | |
| miR-181b-1 | ↑ | |
| miR-181c | ↑ | |
| miR-26a-2 | ↑ | |
| miR-30a | ↑ | |
| miR-3607 | ↑ | |
| miR-365b | ↑ | |
| miR-660 | ↑ | |
| Let-7a-1 | ↑ | |
| Let-7g | ↑ | |
| miR-1291 | ↑ | BAV Aorta |
| miR-143 | ↑ | (+TAA) vs. BAV |
| miR-26a-1 | ↑ | Aorta (no TAA) |
| miR-99a | ↑ | |
| Let-7f-1 | ↑ | |
| Let-7i | ↑ | |
| miR-127 | ↑ | |
| miR-1307 | ↑ | |
| miR-140 | ↑ | |
| miR-146b | ↑ | |
| miR-125b-2 | ↓ | |
| miR-30d | ↓ | |
| miR-100 | ↓ | |
| miR-199a-2 | ↓ | |
| miR-23b | ↓ | |
| miR-423 | ↓ | |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of treating thoracic aortic aneurysm (TAA) in a patient comprising:
a) assaying a blood or plasma sample from a subject diagnosed with TAA for levels of one or more microRNAs selected from the group consisting of miR-1, miR-21, miR-29a, miR-133a, miR-143, and miR-145; one or more MMPs selected from the group consisting of MMP-1, MMP-2, MMP-3, MMP-7, MMP-8, MMP-9, MMP-12 and MMP-13; one or more TIMPs selected from the group consisting of TIMP-1, TIMP-2, TIMP-3, and TIMP-4; or a combination thereof,
b) comparing the levels to control values by multivariate analysis to predict the aneurysm size, and
c) selecting a course of treatment for the patient based on the predicted aneurysm size.

2. The method of claim 1, wherein step c) comprises imaging the patient to measure the size of the aneurysm if the levels predict that the aorta is at least 5 cm in size.

3. The method of claim 2, wherein the patient is imaged by computed tomography (CT), magnetic resonance imaging (MRI), or a combination thereof to measure the size of the aneurysm.

4. The method of claim 1, further comprising surgically treating the TAA in the patient if the aorta is determined to be at least 5 cm in size.

5. The method of claim 1, wherein the control values are levels obtained from a bodily fluid sample from the patient at an earlier time point.

6. The method of claim 1, wherein the control values are based on one or more of a) levels obtained from a bodily fluid sample from a healthy subject or b) levels obtained from a bodily fluid sample from a subject with TAA at least 5 cm in size.

7. The method of claim 1, wherein the microRNA is selected from the group consisting of miR-1, miR-21, miR-29a, miR-133a, miR-143, and miR-145.

8. The method of claim 1, wherein the MMP is selected from the group consisting of MMP-1, MMP-2, MMP-3, MMP-7, MMP-8, MMP-9, MMP-12 and MMP-13.

9. The method of claim 1, wherein the TIMP is selected from the group consisting of TIMP-1, TIMP-2, TIMP-3, and TIMP-4.

10. The method of claim 1, wherein the multivariate analysis comprises analysis of miR-143, MMP-8, and miR-133a levels.

11. The method of claim 1, wherein the multivariate analysis comprises analysis of MMP-2, miR-143, and MMP-8 levels if the subject has a tricuspid aortic valve.

12. The method of claim 1, wherein the multivariate analysis comprises analysis of MMP-2, TIMP-2, miR-143, miR-133a, and miR-145 levels if the subject has a bicuspid aortic valve.

13. The method of claim 1, wherein an at least two-fold decrease in MMP-3 and microRNA-29a levels compared to the control values indicates an increase in aneurysm size if the subject has a tricuspid aortic valve.

14. The method of claim 13, wherein an at least two-fold decrease in MMP-1, MMP-2, MMP-3, MMP-8, MMP-9, TIMP-1, TIMP-2, TIMP-4, and microRNA-29a levels compared to the control values indicates an increase in aneurysm size if the subject has a tricuspid aortic valve.

15. The method of claim 1, wherein an at least two-fold increase in MMP-1 levels and an at least two-fold decrease in TIMP-3 and microRNA-133a levels compared to the control values indicates an increase in aneurysm size if the subject has a bicuspid aortic valve.

16. The method of claim 15, wherein an at least two-fold increase in MMP-1 levels and an at least two-fold decrease in MMP-2, MMP-8, MMP-9, TIMP-1, TIMP-2, TIMP-3, TIMP-4, and microRNA-133a levels compared to the control values indicates an increase in aneurysm size if the subject has a bicuspid aortic valve.

17-43. (canceled)
